(19) United States
(12) Patent Application Publication
(10) Pub. No.: US 2006/0014003 A1
(43) Pub. Date: Jan. 19, 2006

(54) Functional nano-scale gels

(76) Inventors: Matthew R. Libera, New Providence, NJ (US); Svetlana A. Sukhishvili, Maplewood, NJ (US); Peter Krsko, New Brunswick, NJ (US); Ye Hong, Pleasanton, CA (US)

Correspondence Address: MCCARTER & ENGLISH, LLP, FOUR GATEWAY CENTER, 100 MULBERRY STREET, NEWARK, NJ, 07102, US

(21) Appl. No.: 11/141,339

(22) Filed: May 31, 2005

Related U.S. Application Data
(63) Continuation-in-part of application No. 10/626,472, filed on Jul. 24, 2003.

(51) Int. Cl. C12M 3/00

(52) U.S. Cl. 428/195.1

(57) ABSTRACT

Nanometer-scale hydrogels are formed from a polymer film by exposing said film to a focused electron beam of 1 to 10 nm diameter. The hydrogels may be formed in regular patterns, such as arrays, or in irregular patterns. The hydrogels have a plurality of functional groups that can form covalent bonds with proteins while preserving the natural functionality of the proteins. Such functionalized nanohydrogels may serve as a substrate for attachment of other proteins or cells, or may be used in other biological applications.

### CROSS-REFERENCE TO RELATED APPLICATIONS

This application claims the benefit of U.S. Provisional Patent Application No. 60/398,392, filed Jul. 25, 2002, and U.S. Provisional Patent Application No. 60/441,658, filed Jan. 22, 2003, and is a continuation-in-part of U.S. patent application Ser. No. 10/626,472, filed Jul. 24, 2004, all of which applications are incorporated herein in their entirety.

### GOVERNMENT INTERESTS

The development of this invention was supported in part by ARO Grant No. DAAD-10-03-1-0271, awarded by the Army Research Office and NIBIB Grant No. PH1 EB001046-01A1, awarded by the National Institutes of Health. The U.S. Government may have certain rights in this invention.

### FIELD OF THE INVENTION

Polymeric gels, having dimensions in the range of nanometers to microns, for covalent bonding of proteins and adhesion of cells.

### BACKGROUND OF THE INVENTION

The binding of proteins and other biomolecules to surfaces is essential to a variety of applications, including high-throughput proteomic arrays, directed cell adhesion, and biosensors. This creates a need to increase the areal density of protein binding sites on a surface, as well as the need to control cell/surface interactions at subcellular length scales. In addition to the above-stated needs, it is critical to maintain the natural functionality of the protein.

Gels are cross-linked soluble polymers which swell because of their affinity for one or more solvents, but do not dissolve in such solvents due to structural and/or chemical cross-links. Hydrogels are a type of gel that swells in water because of the gel's particular affinity for that solvent. Due to their unique interactions with water, swollen hydrogels can provide near-physiological conditions that preserve the functionality of proteins attached thereto. For biorelevant applications, these properties of hydrogels are important because the control of protein and cell-behavior on synthetic surfaces requires control of the surface chemistry, as well as surface structure at lengths ranging across both the nano-scale and micro-scale.

Surface structures may be created by patterning the surface of a polymer film at the appropriate length scales. In addition to well-established technologies based on photolithography, such surface patterning has been achieved by techniques such as soft lithography, microfluidic patterning, 3-D printing, electron beam patterning, and dip-pen nanolithography, among other traditional and hybrid approaches. Patterning using electron beams has the advantage of enabling the generation of surface-patterned structures with arbitrary shapes and feature sizes as small as a few tens of nanometers.

The co-pending, commonly owned U.S. patent application Ser. No. 10/626,472, filed Jul. 24, 2004, discloses the preparation of hydrogels having surface patterns with dimensions in the micron and nanometer range, and the use of such hydrogels in binding proteins by adsorption and in controlling the placement of cells within the surface patterns. The surface patterns were created using electron-beam patterning or mask patterning of hydrogels formed from a variety of synthetic water-soluble polymers. Said patent application (hereinafter, “the '472 application”) is incorporated herein by reference in its entirety.

The synthetic water-soluble polymers specifically addressed in the '472 application include poly(ethylene glycol) [PEG], poly(ethylene oxide) [PEO], poly(acrylic acid [PAA], poly (methacrylic acid) [PMAA] and poly (hydroxy ethyl methacrylate) [poly-HEMA]. There is little thermodynamic driving force to promote protein adsorption on most hydrogels, and the repelling properties of the surfaces of swollen hydrogels are well known. Even for hydrogels that are densely cross-linked, and thus more hospitable to adsorbed proteins, the adsorption process does not differentiate between the types of proteins that may be present in the environment of the hydrogel. Due to these properties, the adsorption of proteins onto hydrogels is reversible, allowing competition for adsorption sites to displace or translocate desirable proteins over time. It is known that proteins may be covalently bound to hydrogels in such a manner that the functionality of the proteins is preserved. A challenge remains to prepare patterns of protein-functional hydrogels at the nanometer scale, that will retain their functionality across a range of environments. This challenge has been overcome by the invention taught herein.

### SUMMARY OF THE INVENTION

In a first aspect, the present invention includes a patterned gel comprising a polymer film having a superficial pattern including a plurality of nanometer-scale hydrogels. Preferably, the polymer has an exposed functional group that is capable of forming a covalent bond with a type of molecule such as a protein, a polypeptide, an enzyme, a polynucleotide, a polysaccharide, or a bioactive agent. An example of a polymer with such a functional group is a poly(ethylene glycol) with a terminal amine. Further, an additional molecule may be covalently bonded to a molecule that is directly bonded to a hydrogel. In a preferred embodiment, living cells adhere to the hydrogels or molecules bonded thereto.

In a second aspect, the invention comprises one or more hydrogels attached to a common substrate. Each of the hydrogels is selectively functionalized with a different type of molecule. Each type of molecule may be bonded to an array of hydrogels or to a single hydrogel.

In a third variant, the invention comprises a method of making a patterned gel of the first or second aspect. In this variant, a dry polymer film is formed on a substrate by exposed the film to a focused electron beam, which dwells at a single position so as to form a single pixel of exposed polymer film. The film is then hydrated so that the pixel swells to form a nanometer-scale hydrogel. A molecule may then be covalently bonded to the hydrogel.

### BRIEF DESCRIPTION OF THE DRAWINGS

FIG. 1 is an atomic force micrographic image of a pad of a patterned gel according to the present invention, the pad being in its dry state.

FIG. 2 is an atomic force micrographic image of the pad of FIG. 1 while immersed in water.

FIG. 3 is a plot of the height profiles from the images of FIGS. 1 and 2.

FIG. 4 is an atomic force micrographic image of an array of nanometer-scale hydrogels, having an interpixel spacing of 715 nm, in its dry state.

FIG. 5 is an atomic force micrographic image of the array of FIG. 4 in its hydrated, or wet, state.

FIG. 6 is a schematic drawing comparing the height profiles of a row of hydrogels from the array of FIG. 4 to the height profiles of a row of hydrogels from the array of FIG. 5.

FIG. 7 is a graph of the fluorescence intensities of arrays of nanometer-scale hydrogels subjected to different treatments. Photomicrographs of each of the treated arrays are presented along the top of the graph.

FIG. 8 is a plot of the changes in fluorescence intensities of arrays of nanometer-scale hydrogels across a range of interpixel spacings.

FIG. 9 is a profile of fluorescent intensity from single rows from arrays of nanometer-scale hydrogels at various interpixel spacings. Photomicrographs of each of the arrays are presented along the top of the graph.

FIG. 10 is a graph of the fluorescent intensity of two dyes bound to arrays of nanometer-scale hydrogels to which selected proteins have been bound.

FIG. 11 is a reflected light micrographic image of a gel pad exposed to a low electron radiation dose, then subjected to a cell culture procedure.

FIG. 12 is a reflected light micrographic image of a gel pad exposed to an intermediate electron radiation dose, then subjected to a cell culture procedure.

FIG. 13 is a reflected light micrographic image of a gel pad exposed to a high electron radiation dose, then subjected to a cell culture procedure.

FIG. 14 is a micrographic image of a patterned gel showing fibroblasts constrained to adopt specific sizes, shapes and locations by electron-beam patterning.

FIG. 15 is an enlarged view of a first fibroblast of FIG. 14.

FIG. 16 is an enlarged view of a second fibroblast of FIG. 14.

FIG. 17 is an enlarged view of a third fibroblast of FIG. 14.

FIG. 18 is a schematic showing the distribution of radiation dosages applied to a large array of nanometer-scale hydrogels.

FIG. 19 is a reflected light micrographic image of a large array of nanometer-scale hydrogels on a substrate, which have been exposed to high and low radiation dosages as mapped in FIG. 18. The image shows bacteria that have adhered to the substrate and hydrogel array.

FIG. 20 is an atomic force micrographic image of a portion of the large array of FIG. 19, showing pairs of bacteria confined within small areas of the hydrogel array.

FIG. 21 is an array of micrographic images showing bacteria adhering to arrays of nanometer-scale hydrogels having various interpixel spacings and radiation exposures.

FIG. 22 is a schematic image having a geometrically complex pattern adjacent to a micrographic image of a patterned gel formed according to the same complex pattern with nanometer-scale details.

### DETAILED DESCRIPTION OF THE INVENTION

The present invention teaches, among other things, a method for preparing gels that are patterned at nanometer length scales, yet bond covalently with proteins so as to preserve their functionality. The polymers used in such gels contain functional groups that form covalent bonds with biomolecules, such as proteins, upon appropriate chemical treatment. For example, proteins can be covalently bound to PEG in which the terminal hydroxyl group of the PEG is replaced by an amino group [PEG-NH₂]. As taught in the '472 application, the surface patterning of gels may be practiced in a number of variants, each of which is within the scope of the invention, depending on the desired characteristics or uses of the patterned gels. In their most general form, such variants include the steps of selecting a suitable polymer/solvent system, forming a laterally homogenous polymer film on a substrate from a solution of the selected polymer in the selected solvent, exposing selected portions of the polymer film to a radiation source under high vacuum to form a pattern of cross-linked polymer, and removing the unexposed, uncross-linked polymer by washing the film with a solvent. In a preferred embodiment, the pattern and the cross-linking are generated simultaneously using a focused source of radiation, such as a focused electron beam, which can be directed to irradiate a laterally homogeneous polymer film to form the laterally-modulated, two-dimensional pattern of interest. Such methods as are disclosed in the '472 application may also be applied, with some modifications, to the methods of the present invention.

The following examples are explanatory and illustrative of selected variants of the present invention but should not be considered as limiting the scope of the invention. Selected examples from the '472 application are also presented to illustrate aspects of the present invention that are not addressed in the other examples.

### EXPERIMENTAL EXAMPLES

Silicon Substrate Preparation:

Silicon substrates were prepared by cleaving [100] single-crystal wafers (0.5 mm thick) into sections approximately 1 cm×1 cm in area. In a first substrate cleaning process, each substrate was sonicated in acetone for 5 minutes and then in ethanol for 5 minutes. The surface was dried using a nitrogen gas stream. In a second cleaning process, substrates were exposed to UV irradiation from a mercury grid lamp at a maximum power of 450b W for 5 minutes. These substrates were then further exposed to 5% hydrofluoric acid in water for 5 minutes, rinsed in distilled water, exposed again to UV radiation for 5 minutes, and finally exposed to a RF oxygen plasma for 7 minutes. These two substrate cleaning methods led to comparable results when subsequently casting polymer thin films onto them. The second cleaning and preparation process was also used to prepare glass microscope slides for use as substrates. To promote the adhesion of gets, the silicon substrates were further treated by immersion in n-(triethoxysilylpropyl)-o-poly(ethylene oxide) urethane. Gel adhesion was also promoted by immersing the substrates in unsaturated silanes.

Solvent Casting of Polymer Thin Films:

Thin polymer films having thicknesses of about 100 nm were cast by dropping 50 μl of a 1 wt % solution of either monoamine-terminated poly(ethylene glycol) having an average molecular weight of 5000 daltons [PEG-NH₂ 5000] or poly(ethylene glycol) having an average molecular weight of 6800 daltons [PEG 6800] in tetrohydrofuran [THF] onto glass or onto the polished side of the cleaned silicon wafer spinning at approximately 4000 rpm. The silicon substrate was fixed to the spinner either by a vacuum chuck or by double-sided adhesive. After 20 minutes of spinning, the wafer was annealed at 320 K under a vacuum of approximately 50 mTorr for 2 hours.

Electron-Beam Patterning:

Polymer films on silicon substrates were exposed to electron irradiation using a LEO 982 DSM field-emission scanning electron microscope (FEG-SEM) (LEO Electron Microscopy, Thornwood, N.Y.). The vacuum in the specimen chamber during electron irradiation was maintained at approximately 10⁻⁶ Torr. The electron accelerating energy used was 10 keV, and a typical beam current was in the range from 20-100 pA. Because glass is susceptible to electrical charging under electron beam irradiation, gels patterned on glass substrates were typically created and patterned using lower electron energies such a 1-2 keV. The electron beam position and dwell time at each position were controlled using an Emispec Vision data acquisition and control computer system (Emispec Systems, Tempe, Ariz.). Exposure patterns ranging from pixels (i.e., individual points), to lines generated by a linear sequence of points, to square pads generated by a two-dimensional array of exposure points, could all be generated using the scripting capabilities of the Emispec Vision software. Square exposure areas were generated by digitally rastering an electron beam across the polymer surface in a square array of exposure points. An average dose, D, for such an exposure was determined by normalizing the total number of electrons to the total area exposed to electron irradiation: D=(i×t×N)/A where i is the beam current, t is the dwell time per pixel, and N is the number of pixels in the array.

Removal of Insufficiently Cross-Linked Polymer

Removal of insufficiently cross-linked polymer after irradiation corresponds to the development of a resist in conventional photolithography. In the experiments of the present example, irradiation led to cross-linking of the polymer which caused the cross-linked polymer to become insoluble in water or THF. Unirradiated or insufficiently cross-linked polymer was soluble in either of the solvents. Irradiated specimens were developed by washing the specimens in solvent immediately after removing them from the vacuum environment. The specimens were immersed and gently agitated for 5 minutes in 200 ml of THF and then rinsed by immersion in 200 ml of Type 1 water. The developed specimens were then dried under flowing nitrogen gas. Micron-sized particulates were sometimes observed on the surface of some specimens after development, but did not appear to affect the experimental results in a substantive manner.

Morphological Analysis of Surface Patterned PEG and PEG-NH₂

The specimens were studied at four stages during the experimental tests:
(1) after electron-beam exposure; (2) after development of the exposed films; (3) after assessment of the film heights by atomic force microscopy [AFM]; and (4) after protein adsorption studies, using the same LEO 982 FEG SEM that was used to write patterns on the polymer films. Particular care was taken when studying films prior to the development step to minimize the electron dose given to any particular area. Quantitative measurements of film height were made in air as well as in water (pH 5.6) using a Nanoscope IIIa atomic force microscope [AFM] (Digital Instruments—Veeco Metrology Group). Imaging was performed using the AFM with Veeco Nanoprobe tips (model NP-20). The imaging force was minimized to limit deformation of the gels by the AFM tip. Flourescence optical microscopy was done using a Nikon Eclipse E1000 fluorescence optical microscope, and Image Pro-Plus software to quantify the resulting digital fluorescence images.

### Example 1

### Characterization of Patterned PEG Microgels

In Example 1 of the '472 application, patterned gels were characterized for their developmental characteristics (e.g., stability) and swelling properties. A hydrogel was patterned on a film of PEG 6800 by individually irradiating a number of squares at different radiation dosages. Each square was 5.4 μm on a side. At the lower dosages (i.e., less than about 0.2 Coulombs per square meter [C/m²]), no polymer remained on the silicon surface after washing with THF, presumably due to insufficient cross-linking and attachment to the silicon surface. At higher doses (i.e., from 0.214 to 214 C/m²), square pads remained at the silicon surface after washing with THF. One can conclude that, within the higher dose range, electron exposure leads to a net cross-linking effect and attachment to the surface.

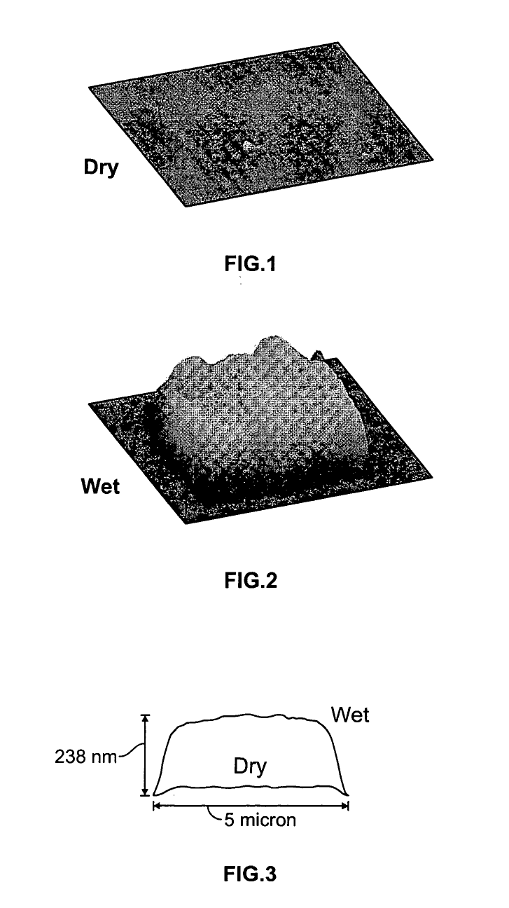

FIGS. 1-3 illustrate the basic swelling phenomenon observed in pads of cross-linked PEG 6800. FIG. 1 shows an AFM image of a pad of PEG 6800 (formed at a dose of 0.306 C/m²) in its dry state after development. The image of FIG. 2 was collected in-situ and shows the same pad in its hydrated state. As shown on the cross-sectional schematic drawing in FIG. 3, the pad height increased from an average of 31.5 nm in the dry state to an average height of 238 nm in the wet state, resulting in a swell ratio of about 7.6, the swell ratio, q, being determined as the ratio of the vertical pad heights: q=h_{wet}/h_{dry}, where h_{wet} and h_{dry} are the wet and dry pad heights, respectively. Also notable, from a comparison among FIGS. 1-3, is that the swelling is highly anisotropic, with little change in the lateral pad dimensions. It is believed likely that this effect is due to the constraints imposed by the binding of the pad to the silicon surface. It was also observed that the average heights of the swollen pads reached a maximum at a dose of about 1.223 C/m², and decreased progressively as the dosages were increased therefrom. This decreased swelling is attributable to increasing degrees of cross-linking within the polymer as the radiation dosage is increased.

### Example 2

### Characterization of PEG-NH₂ Microgels

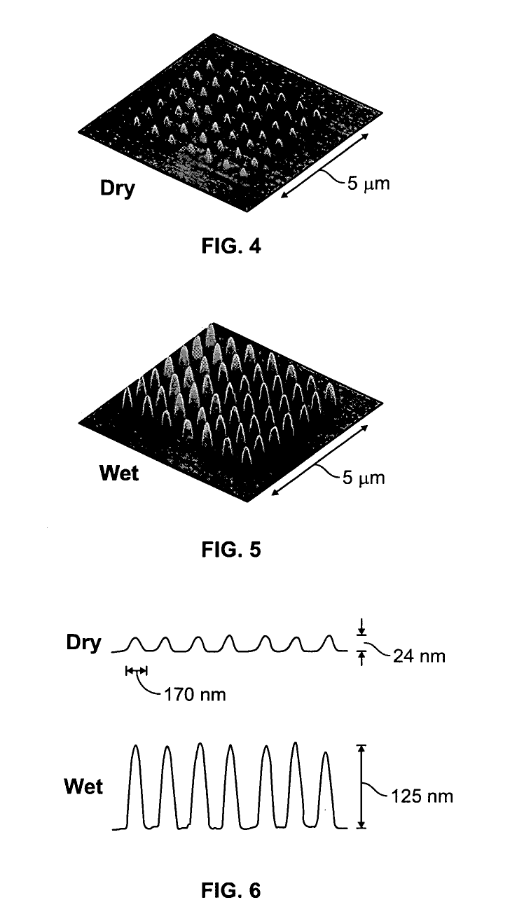

Thin films of PEG-NH₂ 5000 were solvent cast onto cleaned silicon substrates and patterned by electron-beam irradiation, as described above. Similar to the results discussed in Example 1, stable surface-patterned hydrogels were created at dosages ranging from 0.95 to 234 C/m², and the degree of cross-linking increased with the incident dose. It may be noted that the gels studied in Example 1 were formed as clusters of overlapping nanometer-scale hydrogels. In contrast, the nanometer-scale hydrogels of this Example were spaced widely apart so that they could be studied as an array of individual gels. FIGS. 4 and 5 show AFM images of a 5 μm by 5 μm array of 49 individual hydrogels in the dry and wet states, respectively. These hydrogels were created using an electron beam approximately 10 nm in diameter with single-point irradiations (i.e., pixels) separated from each other by 715 nm. For this Example, the beam current was 0.078 picoAmperes (pA) and the dwell time at each position was 125 microseconds (μs). Each hydrogel represents one pixel.

FIG. 6 presents height profiles of the hydrogel array in its dry and wet states. As is shown, the hydrogels swell vertically from a dry height of approximately 24 nm to a wet height of approximately 125 nm. The resulting swell ratio of about 5 is comparable to the swell ratio of about 7.6 observed in Example 1. At a longer dwell time of 209 μs, the swell ratio decreases to about 2.8 (not shown), which is consistent with effects of increased cross-linking observed in Example 1. The lateral dimension of the hydrogel is about 170 nm, which is much larger than the 10 nm beam used to create the pattern. This may be attributable, in part, to the proximity effect, discussed in the '472 application, where electrons are backscattered by the substrate and traverse the polymer film, as well as by scattering of electrons within the polymer film itself. The extent of the proximity effect, and, thus, the lateral dimensions of the hydrogel, can be reduced by using thin-film substrates. The effect of lateral swelling would typically be insignificant since such swelling is constrained by binding of the pads to the silicon surface, similar to the observation made in Example 1.

### Example 3

### Chemical Activity of Functional Groups after Irradiation

The activity of the amine endgroups in the stable nanometer-scale hydrogels was measured using a fluorescein isothiocyanate [FITC] assay, in which the presence of active amine groups would cause the hydrogel to fluoresce green. A series of hydrogel array pads, each having overall dimensions of 5 μm on each side, were formed on a 250 nm spacing at an electron dosage of 2.34 C/m², which results in a swell ratio of about 2. At the spacing used, the individual hydrogels can not be resolved using conventional optical microscopy, so that the array has the appearance of a continuous hydrogel. The array pads were exposed to an 0.05% solution of FITC in sodium carbonate buffer, and held overnight at 4° C. to form thiourea bonds. Unreacted FITC was removed by repeated washings with buffer.

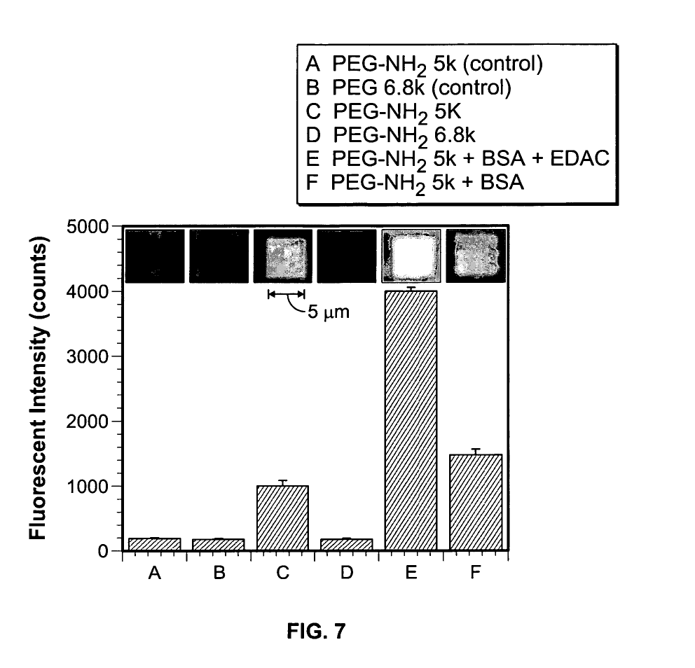

The results of the assays are presented in FIG. 7. Pad C was formed from PEG-NH₂ 5000 and exposed to the FITC assay. As controls to demonstrate the absence of fluorescence, Pads A and B were formed from PEG-NH₂ 5000 and PEG 6800, respectively, but not exposed to FITC. As a control against fluorescence in the absence of amine endgroups, Pad D was formed from PEG 6800 and exposed to the FITC assay concurrently with pad C. The assays of Pads E and F are discussed in Example 5, below. As can be seen in FIG. 7, Pad C fluoresced strongly, while the control pads remained dark. The measured intensity of the fluorescence of pad C was 7.5 times as great as that of the controls, indicating that the amine groups remained chemically active, and were available as protein binding sites. Similar behavior was observed in gel pads exposed to electron doses as high as 95 C/m².

### Example 4

### Functionalization of Nanometer-Scale Hydrogels

Experiments were carried out to determine whether functionalization occurs at the scale of the individual hydrogels. A series of PEG-NH₂ hydrogel array pads were formed at different interpixel spacings across the range of 50 nm to 1250 nm, while holding the dwell time at a constant value. The activity of the amine endgroups in the hydrogels was measured using the FITC assay of Example 3. The fluorescent intensity of each array was measured over an area of 5 μm by 5 μm. The experiment was replicated five times on two different silicon substrates.

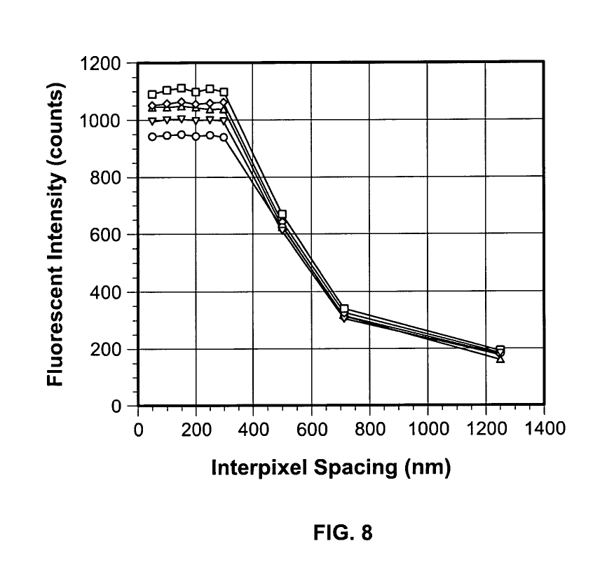

The results of the replicate assays are plotted in FIG. 8. It can be seen that the fluorescent intensity across the measured area is constant for interpixel spacings of 300 nm and below, despite the fact that the total dose across the same area increases by a factor of 36 as the interpixel spacings are decreased from 300 nm to 50 nm. This suggests that the total number of accessible amine groups remains roughly constant over a range of irradiation dosages, once the hydrogels begin to overlap and form laterally continuous structures. The variation of intensity across the five replicate trials was less than 10% of the average, which suggests a level of reproducibility sufficient to make robust devices.

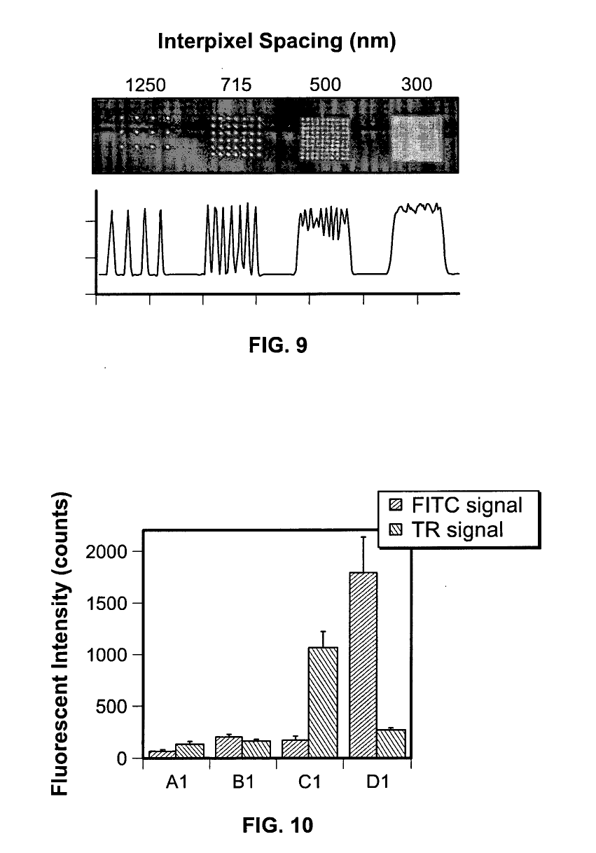

FIG. 8 shows that the absolute fluorescent intensity of the hydrogel array decreases as the interpixel spacings increase beyond about 300 nm. Increasing the spacing simply results in less fluorescing material per unit area. This observation is supported by the results shown in FIG. 9, which shows fluorescence micrographs of hydrogel arrays having interpixel spacings of 1250 nm, 715 nm, 500 nm and 300 nm, respectively, and intensity readings for the hydrogels in the arrays. It can be seen that the peak fluorescence intensities of the individual hydrogels, visualized at interpixel spacings of 1250 nm, 715 nm and 500 nm, is about the same as that of the overlapping array hydrogels visualized at interpixel spacings of 300 nm or less.

### Example 5

### Amplification of Amine Concentrations

Bovine serum albumin [BSA] was bound covalently to the amine groups of the PEG-NH₂ 5000 hydrogel to amplify the concentration of such groups. A PEG-NH₂ 5000 hydrogel array pad was exposed to a solution of 0.1% BSA with freshly prepared 1-ethyl-3-(3-dimethylaminopropyl)carbodiimide hydrochloride [EDAC]. An identical array pad was exposed to 0.1% BSA without EDAC. Both samples were kept at room temperature for 2 hours, then washed repeatedly with 0.1 M phosphate buffer saline, then with 1 M NaCl buffer solution to remove adsorbed BSA. The pads were then allowed to react with FITC solution overnight at 4° C.

The results of this treatment are presented as Pads E and F of FIG. 7, which may be compared to the results presented for Pad C. It may be recalled from Example 2, that the fluorescent intensity of Pad C results from the binding of FTIC to the amine endgroups of the PEG-NH₂ polymer, without amplification by a bound protein. It can be seen that Pad E exhibits a fluorescent intensity that is more than four times greater than that of the unamplified Pad C, indicating that the BSA is covalently bound to the amine groups of the hydrogel. It can also be seen that the intensity exhibited by Pad F is comparable to that exhibited by Pad C, indicating that the extent of BSA adsorption is low.

### Example 6

### Selective Functionalization of a Hydrogel Pad

Separate hydrogel arrays were spotted with fibronectin (Fn) and laminin (Ln) to demonstrate that the hydrogels could be selectively functionalized on a single substrate. Four array pads of BSA-amplified hydrogels were patterned on a single silicon wafer, each pad having lateral dimensions of 5 μm on each side and hydrogels spaced at an interpixel spacing of 100 nm. An aqueous solution of a photoactivatable heterobifunctional cross-linker (sulfosuccinimidyl-6-[4′-azido-2′-nitrophenyamino] hexanoate [sulfo-SANPAH]) was pipetted onto the wafer, and allowed to react in the dark for 30 minutes. Ln was spotted onto one of the pads, Fn was spotted on another, and PBS was spotted onto the two remaining pads which served as controls. The entire wafer was then exposed to UV light to bind the Ln and Fn to the BSA via the sulfo-SANPAH cross-linker.

The proteins were then assayed to confirm that the different pads were functionalized with different proteins. The wafer was first exposed to a solution containing mixed primary antibodies of (1) anti-rabbit Ln and (2) anti-human Fn, followed by normal donkey serum [NDS] blocking. The wafer was then exposed to a solution of mixed secondary antibodies of (1) donkey anti-rabbit immunoglobulin-G bound to FITC [IgG-FITC] with minimum cross-reaction to rabbit; and (2) donkey anti-human Fn immunoglobulin-G bound to Texas Red [IgG-TR] with minimum cross-reaction to human. The fluorescent intensities of FITC and TR were then measured for each pad. The results of the assays are shown in FIG. 10, in which Pad C1 is the pad with bound Ln, Pad D1 is the pad with bound Fn, and Pads A and B are the controls. Background signals due to non-specific binding have been subtracted from the absolute values to produce the values shown in the figure. It can be seen that the pads with bound proteins fluoresced much more strongly than the control pads, at ratios of about 8 for Ln and about 9 for Fn. These results show that Ln and Fn are selectively attached to different hydrogel pads with high differentiation.

### Example 7

### Control of Cell Adhesion on PEG and Poly-HEMA Films

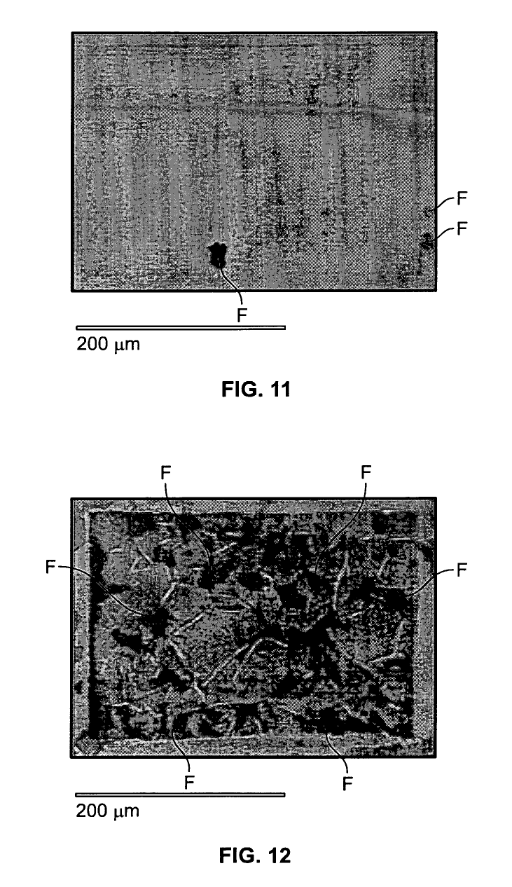

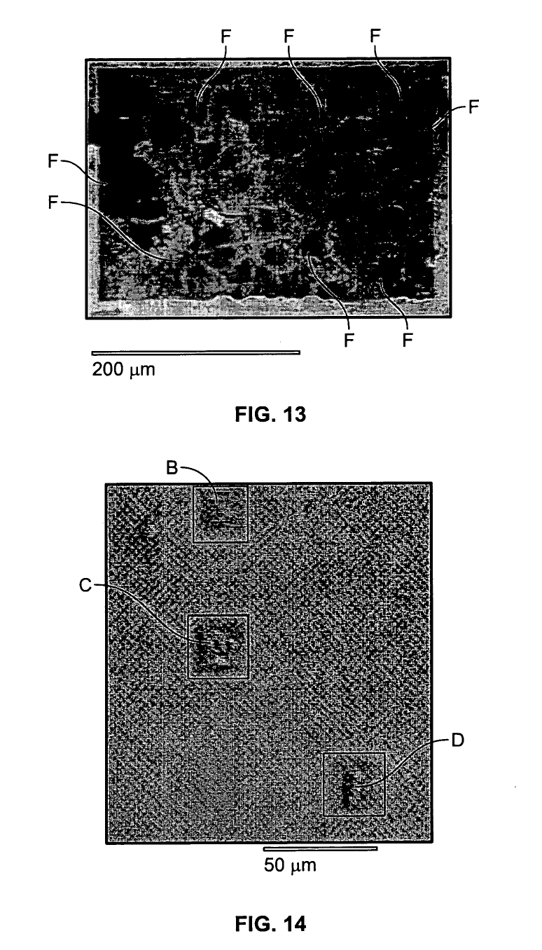

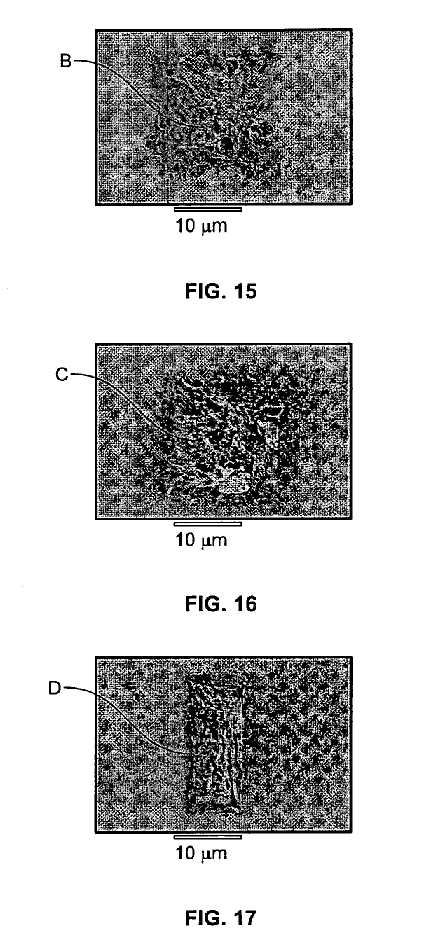

The examples in the '472 application revealed that proteins, such as Fn, did not adhere well to hydrogels that had been exposed to lower doses of radiation, but adhered extensively to hydrogels that had been exposed to higher doses of radiation, and thus were more densely cross-linked. Further testing demonstrated that cells also adhered more strongly to densely cross-linked hydrogels. A thin film of PEG 6800 was solvent cast onto a silicon substrates and three pads, each approximately 200 μm by 300 μm in size, were created using incident radiation doses which sampled conditions in the ranges from those where Fn would not adsorb at all to those where Fn would adsorb extensively. Cells were seeded on the hydrogels and substrate and incubated following procedures which are explained in detail in the '472 application. As illustrated by FIG. 11, fibroblasts F did not adhere to the PEG surface exposed to lower electron doses, specifically, 0.5 C/m², whereas increasing numbers of fibroblasts F adhere under conditions of higher electron doses, specifically, 5 C/m² (FIG. 12) and 20 C/m² (FIG. 13). At the highest doses studied, a confluent layer of fibroblasts F was created (see FIG. 13). By laterally modulating the electron dose and exploiting the patterning capabilities of the electron-beam irradiation method disclosed herein, polymer films of poly-HEMA were patterned to create specific locations where individual cells adhered to the film surface. FIGS. 14-17 show that electron-beam patterning of poly-HEMA on silicon can be used to control the size, shape, and relative positions of fibroblasts on the polymer film. FIG. 14 is an optical micrograph of a poly-HEMA film in which areas B, C and D have been treated by electron-beam patterning at incident doses of 0.1 C/m², 0.3 C/m², and 0.25 C/m², respectively. FIGS. 15, 16 and 17, respectively, are enlarged views of the fibroblasts that adhere to areas B, C and D. Examination of FIGS. 14-17 shows that the fibroblasts have been constrained to adopt the specific sizes, shapes and locations of the three areas subjected to electron-beam patterning.

### Example 9

### Cell Adhesion on Nanometer-Scale PEG Hydrogels

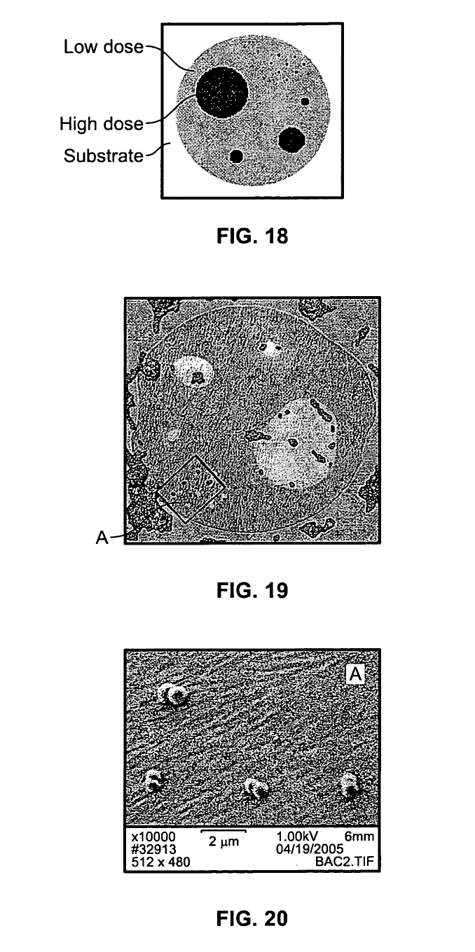

As shown in the '472 application and discussed herein, the size, shape, and position of hydrogel pads can all be controlled by the lateral modulation of dose and spatial resolution of the focused electron beam irradiation. Therefore it should also be possible to control the size, shape and position of the adhesive regions. Extending this concept to the finest length scales, one can expect that the adhesive property of the square pad can itself be modulated to, for example, control the specific locations and sizes of focal adhesions between the cell and the substrate below it, with spatial resolution at the micron and nanometer scale. One demonstration of such control is shown in FIGS. 18-20 with regard to the growth of the bacterium Staphylococcus epidermidis. FIG. 18 is a diagram of a large array PEG hydrogels, at 100 nm interpixel spacings, that has been formed on a glass substrate at different levels of electron-beam radiation. Most of the array has been formed at a low dose of radiation, therefore it is expected that bacteria would not adhere to the hydrogels so formed. Smaller areas within the larger array have been formed at higher doses, in the expectation that bacteria would adhere to the more highly cross-linked hydrogels. These expectations may be confirmed through examination of FIG. 19, which shows the distribution of bacteria after they have been cultured on the substrate. The area exposed to low doses of radiation is substantially clear of bacteria. Small clusters of bacteria are present in the areas that have been exposed to higher doses of radiation, and are substantially confined to those areas. Area A, which is shown in an enlarged view in FIG. 20, consists of a field of very small high-dose areas, each of which is roughly 1 μm across, and thus would contain roughly 70-100 hydrogels. Four of these areas are each occupied by only one pair of bacteria, each pair being confined entirely within its respective area. This suggests that small cellular organisms, such as bacteria, can be effectively confined within relatively small groups of hydrogels, which may be placed at any position on a substrate. It may also be noted that the bacterial clusters present on the hydrogels are much smaller than those on the exposed substrate, suggesting that expansion of the clusters is inhibited by their attachment to the hydrogels.

### Example 10

### Dependence of Bacterial Cluster Size on Hydrogel Spacing

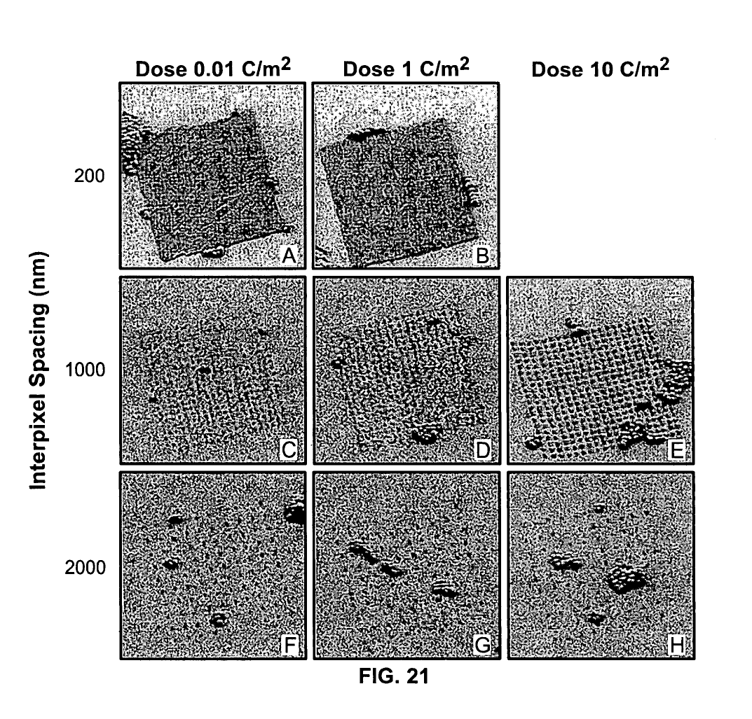

FIG. 21 represents the dependency of bacterial cluster size on the spacing between hydrogels. Eight PEG hydrogel array pads (i.e., Pads A-H) were prepared on a substrate and S. epidermidis bacteria were cultured thereupon. The pads and substrate were then washed to remove non-adhering bacteria. The remaining bacteria were fixed by conventional processes and examined by optical microscopy. At an interpixel spacing of 200 nm (i.e., Pads A and B), at which the hydrogels form a continuous layer, both Pad A, which was exposed to a radiation dose of 0.01 C/m², and Pad B, which was exposed to a dose of 1 C/m², are clear of bacteria. At an interpixel spacing of 1000 nm (i.e., Pads C, D and E), a small number of bacteria adhere to the hydrogels, with the number of such bacteria increasing as the radiation dosages are increased. At an interpixel spacing of 2000 nm, larger clusters of the bacteria adhere to the pad, with the largest clusters observed on Pad H, which was exposed to the highest dose of radiation.

### Example 11

### Formation of Arbitrary Patterns at Micron and Nanometer Scales

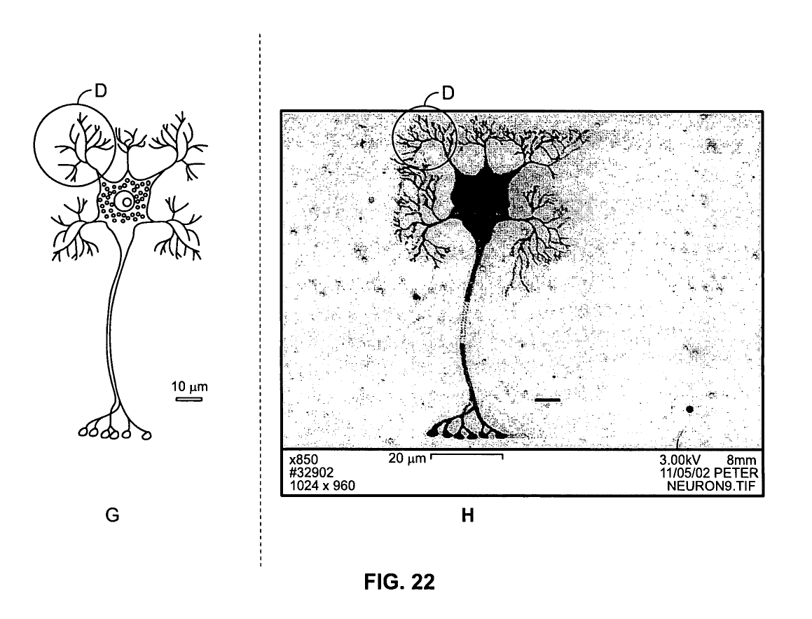

FIG. 22, which has been adapted from the '472 application, shows an optical micrograph of a film of poly(desamino tyrosyl-tyrosine ethyl ester carbonate) [poly DTE carbonate] on a silicon substrate (frame H) that has been electron-beam patterned using a schematic image of a neuron (frame G) using the focused electron-beam method of the present invention. Although poly(DTE carbonate) does not form hydrogels, it does form gels in other solvents that can be manipulated in the same manner as hydrogels. The schematic image of the neuron has successfully been reproduced at the length scale of the cell itself. It may be observed that the dendrites D of the surface pattern have been reproduced at a nanometer scale. Thus, the method of the present invention provides the ability to create such finely scaled patterns, at micron and nanometer scales, together with the ability to control the amount of protein locally adsorbed onto the patterned as demonstrated in Examples 4-6.

The present invention is directed to the creation of surface-patterned nanometer-scale hydrogels, in particular those formed from PEG or PEG-NH₂, though not limited thereto, for controlled adsorption of proteins, covalent binding of proteins and other molecules to surfaces, and adhesion of cells. The enhanced spatial resolution can be exploited to create patterns with characteristic length scales relevant to cellular and sub-cellular processes. The hydrogels may be created in arrays or in other patterns or irregular arrangements desired by the user. The use of PEG-NH₂, or other polymers having exposed functional groups, allows proteins to be covalently bound to the hydrogel, preventing the proteins from being dislodged due to competition from undesirable proteins and chemical compounds. Further, various proteins can be selectively bound to different array pads, promoting the use of the treated pads in processing multiple analytes. The process can also be used to precisely locate the adhesive junction between cells and a substrate and to confine cell growth within defined areas of the substrate. Cells, including bacteria, can be contained within small fields of hydrogels, individually, in pairs, or in larger clusters.

The novelty and unobviousness of the invention derive from several factors including, but not limited to, combinations of hydrogel properties. The nanometer-scale hydrogels are formed from thin films of solid uncross-linked polymers subjected to electron irradiation under vacuum. Proteins can be covalently bound to such nanometer-scale hydrogels, or to microscale hydrogels, to amplify the number of functional groups, or to participate in processes such as immunoassays or chemical detection. Further, other proteins may be covalently bound to the functional groups of the first protein. Protein-functional hydrogels so formed are effective at nanometer-scale lengths. Other potential applications include the selective functionalization of individual hydroge's at the nanometer-scale using dip-pen nanolithography systems.

Although the invention disclosed herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the invention as defined by the appended claims. Variations of the foregoing embodiments, particularly with respect to selection of materials (e.g., polymers, solvents, proteins or cell types), methods of forming, patterning and cross-linking the polymer films, environmental conditions for implementing the foregoing embodiments and their variants, and the like, which would become apparent to one skilled in the art upon reading this disclosure, are to be considered as part of this invention.

Those practitioners having ordinary skill in the relevant arts, particularly in those arts relevant to the use of polymer surfaces for the control of protein and cell adhesion, will recognize that a broad range of polymers can be used to create patterned nanometer-scale hydrogels under conditions and using methods taught herein. For example, hydrogels capable of covalently bonding to proteins or other chemical moieties may be made from polymers having any exposed functional group that can be made to form such bonds. Such functional groups may include one or more of the following: (1) amine groups; (2) carboxyl groups; (3) thiol groups; (4) maleimide groups; (5) epoxide groups; (6) acrylate groups; and (7) hydroxyl groups. Such functional groups may be at the terminal ends of the polymer backbone, may extend as side groups off of the polymer backbone, or may be within the polymer backbone itself. A variety of polymer backbones are suitable for forming the hydrogels of the present invention, including the following: (1) PEG; (2) PEO; (3) PAA; (4) PMAA; and (5) poly(HEMA); or combinations thereof. The hydrogels may be formed from homopolymers or co-polymers.

The ordinarily-skilled practitioner will also recognize that patterns may be formed on polymer films by methods other than the use of a focused electron beam on a homogenous, extensive polymer film. For example, a surface pattern may be formed on a polymer film may be formed before the cross-linking of the polymer is initiated. Films of uncross-linked polymer may be patterned directly onto a surface at micron or nanometer dimensions using, for example, stamping, printing, writing, or confinement techniques. The entire surface of the patterned area would then be exposed to radiation to effect the cross-linking of the polymer.

It should also be noted that, although the Examples disclosed herein address the covalent bonding of fibronectin, laminin, and bovine serum albumin, the skilled practitioner will recognize that the present invention may be extended to other proteins and other molecules such as polypeptides, enzymes, polynucleotides, polysaccharides, and bioactive agents. Moreover, the skilled practitioner will be able to adapt the present invention to control the adhesion of cells other than fibroblasts, particularly those cell lines, such as endothelial cells or neuronal cells, which tend to adhere to and spread on both natural and synthetic surfaces. The skilled practitioner will also recognize that the invention may also be adapted to control the adhesion of bacteria other than staphylococci, as well as other single celled organisms. It is also worth noting that the ability to form finely scaled, irregular patterns, together with the ability to control the amount of protein bound to the patterned suggest radically new applications in the area of directed cell growth such as that associated with axonal regeneration and synapse formation in the central and peripheral nervous systems.

We claim:

1. A patterned gel, comprising a film made at least in part from a polymer, said film having a first portion with a superficial pattern and a second, non-patterned portion, said first portion including a plurality of nanometer-scale hydrogels and being distinguishable from said second portion by a physcial property.

2. The patterned gel of claim 1, wherein at least some of the hydrogels of said plurality of hydrogels overlap one another.

3. The patterned gel of claim 1, wherein said polymer has an exposed functional group on a polymer backbone said functional group being capable of forming a covalent bond with a molecule.

4. The patterned gel of claim 3, wherein said functional group is a terminal group.

5. The patterned gel of claim 3, wherein said functional group is a side group.

6. The patterned gel of claim 3, wherein said functional group is within said polymer backbone.

7. The patterned gel of claim 3, wherein said exposed functional group is selected from the group consisting of an amine group, a carboxyl group, a thiol group, a maleimide group, an epoxide group, an acrylate group, and an hydroxyl group.

8. The patterned gel of claim 3, wherein said molecule is selected from the group consisting of a protein, a polypeptide, an enzyme, a polynucleotide, a polysaccharide, and a bioactive agent.

9. The patterned gel of claim 3, wherein said molecule is a protein.

10. The patterned gel of claim 3, wherein said molecule is a dendrimer.

11. The patterned gel of claim 1, wherein the hydrogels of said plurality of hydrogels are arranged in a regular pattern.

12. The patterned gel of claim 11, wherein regular pattern is an array.

13. The patterned gel of claim 1, wherein the hydrogels of said plurality of hydrogels are irregularly arranged.

14. The patterned gel of claim 3, wherein said polymer backbone is selected from the group consisting of a poly(ethylene glycol), a poly(ethylene oxide), a poly(acrylic acid), a poly(methacrylic acid), a poly(hydroxyethylmethacrylate), and poly(desamino tyrosyl-tyrosine ethyl ester carbonate), and co-polymers thereof.

15. The patterned gel of claim 1, wherein said polymer is a homopolymer.

16. The patterned gel of claim 1, wherein said polymer is a copolymer.

17. The patterned gel of claim 1, wherein said film has a first layer which includes a first polymer, and a second layer, which includes a second polymer.

18. The patterned gel of claim 1, wherein said first portion of said film exhibits a first degree of swelling upon exposure to a solvent and said second portion of said film exhibits a second degree of swelling upon exposure to said solvent, said second degree of swelling being different than said first degree of swelling, whereby said degrees of swelling represent said physical property.

19. The patterned gel of claim 1, wherein said first portion of said film exhibits a first chemical activity and said second portion of said film exhibits a second chemical activity which is different than said first chemical activity, whereby said chemical activities represent said physical property.

20. The patterned gel of claim 1, further comprising a first protein covalently bonded to said plurality of nanometer-scale hydrogels.

21. The patterned gel of claim 20, further comprising a second protein covalently bonded to said first protein.

22. The patterned gel of claim 20, further comprising a cell adhered to said first protein.

23. The patterned gel of claim 21, further comprising a cell adhered to said second protein.

24. The patterned gel of claim 1, wherein said first portion of said film includes an adhesive zone and a non-adhesive zone, cells adhering to said adhesive zone in preference over said non-adhesive zone.

25. The patterned gel of claim 24, further comprising a cell adhering to said adhesive zone.

26. The patterned gel of claim 25, wherein said adhesive zone has a lateral extent on the order of the size of said cell.

27. A patterned gel, comprising a film made at least in part from a polyethylene glycol having a terminal amine group, said film having a plurality of nanometer-scale hydrogels formed thereon.

28. The patterned gel of claim 27, wherein the hydrogels of said plurality of hydrogels are arranged in a regular array.

29. The patterned gel of claim 28, further comprising a first molecule covalently bonded to at least one of the hydrogels of said plurality of hydrogels.

30. The patterned gel of claim 29, further comprising a second molecule covalently bonded to said first molecule, said second molecule selected to influence the adhesion of cells to said gel.

31. The patterned gel of claim 30, further comprising a cell confined within an area defined by said plurality of nanometer-scale hydrogels.

32. A patterned gel, comprising a plurality of nanometer-scale hydrogels, at least one of said hydrogels having a first molecule bonded thereto and at least one other hydrogel having a second molecule covalently bonded thereto.

33. The patterned gel of claim 32, wherein said first molecule has a third molecule covalently bonded thereto.

34. The patterned gel of claim 31, wherein said first molecule is different than said second molecule.

35. A method of making a patterned gel, comprising the steps of:
forming a dry polymer film on a substrate; and
exposing a portion of the dry polymer film to a focused electron beam, which dwells at a single position so as to form a single pixel of exposed polymer film.

36. The method of claim 35, said focused electron beam having a characteristic diameter of from about 1 nanometer to about 10 nanometers.

37. The method of claim 35, further comprising the step of hydrating said dry polymer film so that said pixel swells to form a nanometer-scale hydrogel.

38. The method of claim 37, further comprising the step of covalently bonding a molecule to said nanometer-scale hydrogel.

39. The method of claim 35, wherein said electron beam dwells at a plurality of positions over said portion of the dry polymer film so as to form a plurality of pixels of exposed polymer film.

40. The method of claim 39, wherein the intensity of said electron beam is modulated at each of the positions of said plurality of positions.

41. The method of claim 40, wherein the intensity of the electron beam is varied along a dimension parallel to a surface of said dry polymer film.
